(19) United States
(12) Patent Application Publication
(10) Pub. No.: US 2008/0276940 A1
(43) Pub. Date: Nov. 13, 2008

(54) Breathing-Gas Delivery System With Exhaust Gas Filter Body And Method Of Operating A Breathing-Gas Delivery System

(76) Inventors: Bradley P. Fuhrman, Buffalo, NY (US); Mark Dowhy, West Seneca, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 12/117,512

(22) Filed: May 8, 2008

Related U.S. Application Data
(60) Provisional application No. 60/916,667, filed on May 8, 2007.

(51) Int. Cl. A61M 16/00; A62B 7/10

(52) U.S. Cl. 128/204.26; 128/204.18; 128/205.12

(57) ABSTRACT

Breathing-gas delivery systems and methods are described. In one such system there is a movable partition, a housing and a filter body. The housing is disposed about the movable partition, and thereby provides a respirator side on a first side of the partition, and a patient side on a second side of the partition. The housing also has (a) a patient inspiration orifice on the patient side, which is adaptable to supply breathing-gas to a patient, and (b) a gas exhaust orifice on the patient side.

### CROSS-REFERENCE TO RELATED APPLICATION

This application claims the benefit of priority to U.S. provisional patent application Ser. No. 60/916,667, filed on May 8, 2007.

### FIELD OF THE INVENTION

The present invention relates to patient ventilators, including re-breathing devices. The present invention may be embodied as a scavenger system associated with a patient ventilator, which may be used to administer a therapeutic agent in gas or particulate form.

### BACKGROUND OF THE INVENTION

In the prior art, a patient ventilator is a device that moves air into and out of a patient's lungs. A re-breathing system may be associated with a ventilator, and used to allow a patient to inhale gas that was previously exhaled. Such systems are particularly useful when the inhalation gas includes a therapeutic agent. However, mechanisms must be provided in such systems to allow re-breathing gas to leave the system, either to make room for new gas or to protect against overpressuring a patient's lungs. Rebreathing gas that exits the system may be harmful or dangerous to people, such as medical workers, near the system. To avoid this, some systems exhaust directly to a hospital suction line, though doing so may subject the system to negative pressure of the suction line, which may adversely impact performance of the system. Consequently, there is a need for a ventilating system, and a rebreathing system in particular, that will minimize the venting of exhaled gas and particulates such as infectious droplets into the area around the system, and will protect the re-breathing system from pressure perturbations due to the waste disposal suction line.

### SUMMARY OF THE INVENTION

The invention may be embodied as a patient ventilator system having a housing with a gas exhaust orifice and a filter body. The filter body may have (a) an inlet connected to the gas exhaust orifice, (b) an outlet connected to a suction line, (c) a major-through-passage for carrying exhaust gas from the gas exhaust orifice to the suction line, which may be accomplished without crossing a porous barrier of the filter body, and wherein the filter body is capable of passing gas from outside the ventilator across the porous barrier to the major-through-passage. In one such system, the filter body is tubular and the major through-passage is formed at least in part by the porous barrier. In another system according to the invention, the filter body includes a first channel, a second channel and a third channel, and (a) the first channel may be connected to receive exhaust gas from the gas exhaust orifice, (b) a porous body, may be attached to the second channel, (c) the first channel merges with the second channel at a merger location, and (d) the third channel may extend from the merger location to a waste line.

The invention may be embodied as a breathing-gas delivery system designed for re-breathing. In such a system there may be a movable partition, a housing and a filter body, which may be tubular. The housing is disposed about the movable partition, and thereby provides a respirator side on a first side of the partition, and a patient side on a second side of the partition. The housing also has (a) a patient inspiration orifice on the patient side, which is adaptable to supply breathing-gas to a patient, and (b) a gas exhaust orifice on the patient side.

The tubular filter may have (a) an inlet connected to the gas exhaust orifice, (b) an outlet connected to a suction line, and (c) a major-through-passage for carrying exhaust gas from the gas exhaust orifice to the suction line without crossing the filter. The tubular filter may be capable of passing gas from outside the rebreather through the filter to the major-through-passage. An exhaust line may be used to connect the tubular filter inlet to the gas exhaust orifice of the housing.

In lieu of a tubular filter, a rebreathing system according to the invention may utilize a filter body that includes a first channel, a second channel and a third channel. The first channel may be connected to receive exhaust gas from the gas exhaust orifice, (b) a filter, which may be a porous body, may be attached to the second channel, (c) the first channel merges with the second channel at a merger location, and (d) the third channel may extend from the merger location to a waste line.

A valve may be positioned to regulate flow of exhaust gas through the major-through-passage of the filter body. For example, the valve may allow exhaust gas to flow through the major-through-passage and hence out of the patient side of the housing when the pressure on the patient side of the housing exceeds the pressure on the respirator side of the housing. Further, the valve may be used to prevent exhaust gas from flowing through the major-through-passage when the pressure on the patient side of the housing is less-than the pressure on the respirator side of the housing. Or, the valve may be a control valve arranged to selectively inhibit the flow of exhaust gas if a pressure difference between the respirator side and the patient side is not within a range of acceptable pressures. Other means may be provided to open and close this valve in such a way that exhaust occurs only during a desired portion of exhalation.

A system according to the invention may include an exhaust gas filter positioned to filter materials carried by the exhaust gas. The exhaust gas filter may be positioned between the gas exhaust orifice and the tubular filter inlet, or may be positioned to filter exhaust gas that has passed through the major-through-passage of the filter body.

The invention may be embodied as a method of delivering breathing-gas. In one such method, a breathing-gas delivery system is provided. For example, the system may be like that described above. The partition may be moved in order to cause inspiratory gas to leave the patient side via the patient inspiration orifice to supply breathing-gas to a patient. The partition may also be moved in order to allow the patient side of the housing to receive exhaled gas from the patient. If the pressure on the patient side is not at a desirable level, then gas may be allowed to leave the patient side via the gas exhaust orifice, thereby providing exhaust gas to the filter body. The exhaust gas may be passed through the major-through-passage. Before, during and after passing the exhaust gas through the major-through-passage, gas from outside the system may be passed through a filter to the major-through-passage. In addition, the exhaust gas may be passed through a gas exhaust filter before entering a suction line, or other waste disposal line that is suitable for handling exhaust gas.

Movement of the partition may be effected by a respirator that is in pneumatic communication with the respirator side of the housing. Movement of the partition during inhalation may be effected by increasing the pressure on the respirator side, and movement of the partition during exhalation may be effected by decreasing the pressure on the respirator side.

### BRIEF DESCRIPTION OF THE DRAWINGS

For a fuller understanding of the nature and objects of the invention, reference should be made to the accompanying drawings and the subsequent description. Briefly, the drawings are:

FIG. 1 is a schematic of a system according to the invention;

FIG. 2A depicts a valve in the open position;

FIG. 2B depicts a valve in the closed position;

FIG. 3 depicts a filter body having an exhaust gas filter;

FIG. 4 depicts a cross-sectional view of the filter body, taken along the line 4-4 of FIG. 3;

FIG. 5 depicts an end view of the exhaust gas filter shown in FIG. 3;

FIG. 6 is a flow diagram of a method according to the invention; and

FIG. 7 depicts another type of filter that may be used in the invention.

### FURTHER DESCRIPTION OF THE INVENTION

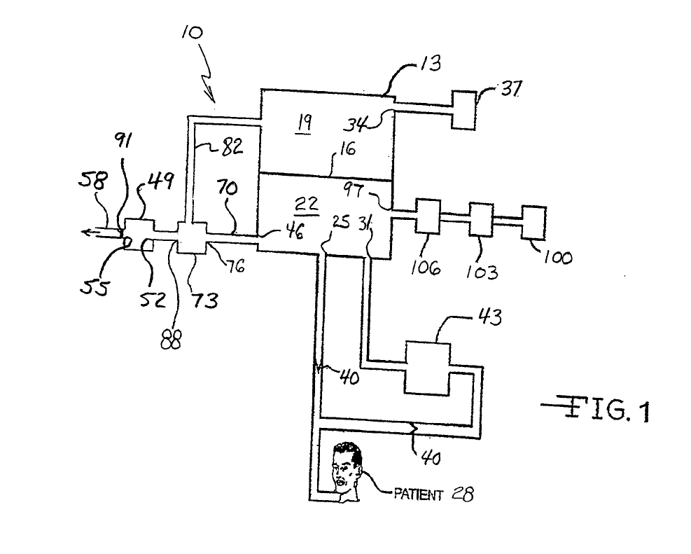

FIG. 1 is a schematic of a system 10 according to the invention. In FIG. 1 there is shown a housing 13 and a movable partition 16. The housing 13 is disposed about the movable partition 16, and the movable partition 16 divides the housing 13 into a respirator side 19 on a first side of the partition 16, and a patient side 22 on a second side of the partition 16. The housing 13 has a patient inspiration orifice 25 on the patient side 22, which may be adaptable to supply breathing-gas to a patient 28. The housing 13 may also have a patient return orifice 31, which may operate in conjunction with the patient inspiration orifice 25 to convey patient side 22 gas to and from the patient 28, thereby facilitating inhalation and exhalation.

In use, the partition 16 may be moved in order to facilitate inhalation and exhalation by the patient 28. The system 10 may be arranged to be especially well suited for delivering respiratory gases to and from a patient 28 while allowing rebreathing of gas on the patient side 22. Rebreathing may be beneficial in order to provide a proper dose of a medicament to the patient 28 and/or to conserve medicaments, particularly when the medicament is costly.

The housing 13 may be equipped with a respirator orifice 34 on the respirator side 19. The respirator orifice 34 may be in pneumatic communication with a respirator 37, and the respirator 37 may control the pressure on the respirator side 19 of the housing 13 in order to control when and how gas from the patient side 22 is delivered to and/or taken from the patient 28. For example, by increasing the pressure on the respirator side 19, the respirator 37 may cause the movable partition 16 to move toward the patient 28, thereby causing gas to leave the patient side 22 via the patient inspiration orifice 25 in order to supply breathing gas to the patient 28. Also, by decreasing the pressure on the respirator side 19, the respirator 37 may cause the movable partition 16 to move away from the patient 28, thereby causing gas to enter the patient side 22 via a return orifice 31 in order to accept exhaled gas from the patient 28. Check valves 40 may be included in the system 10 in order to assure that inhaled gas and exhaled gas are properly conveyed to and from the patient side 22 of the housing 13. Furthermore, a CO₂ scrubber 43 may be included to remove CO₂ from gas on the patient side 22 of the housing 13.

The housing 13 also has a gas exhaust orifice 46 on the patient side 22. The system 10 shown in FIG. 1 has a tubular filter body 49 having an inlet 52 connected to the gas exhaust orifice 46. The tubular filter body 49 may be a cylindrical tube, or may be another shape, including standard shapes such as a square, oval, or triangle. The tubular filter body 49 also has an outlet 55 connected to a suction line 58. The suction line 58 may be a vacuum line, which is commonly found in most hospitals.

The filter material of the tubular filter body 49 provides a porous barrier 61 that is disposed away from a central axis 64 of the tubular filter body 49 in order to form a major-through-passage 67 for carrying exhaust gas from the gas exhaust orifice 46 to the suction line 58 without crossing the porous barrier 61 of the tubular filter body 49. The porous barrier 61 of the tubular filter body 49 is able to pass gas from outside the rebreather system 10 across the filter 49 to the major-through-passage 67, and thereby prevent harmful liquid or solid particulate substances in the exhaust gas from leaving the system 10 via the tubular filter body 49. When the suction line 58 supplies a pressure that is below the pressure of the gas outside the system 10, gas from outside the system 10 will cross the porous barrier 61 and ultimately enter the suction line 58. In many hospitals, the suction line 58 supplies a pressure of −30 centimeters of water, and in that situation it is believed the porous barrier 61 may acceptably have a pressure drop of up to one centimeter of water, but preferably should be less than 0.5 centimeters of water.

When the suction line 58 supplies a pressure that is below the pressure of the patient side 22, gas from the patient side 22 may be exhausted from the patient side 22 via the gas exhaust orifice 46, and then may be supplied to the suction line 58 after passing through the major-through-passage 67. When exhaust gas is moved from the patient side 22 into the major-through-passage 67, the exhaust gas will be mixed with gas that has crossed the porous barrier 61 of the tubular filter body 49 from outside the system 10. The mixed gas will then proceed to the suction line 58. In order to prevent particulates, viruses, bacteria and/or medicament from crossing the porous barrier 61 of the tubular filter body 49 to outside the system 10, the porous barrier 61 may be a HEPA filter and/or the flow rate of gas crossing from outside the system 10 into the major-through-passage 67 may be kept sufficiently high.

It is believed that the pressure drop across the porous barrier 61 of the tubular filter body 49 should be minimal so that inflow of gas across the porous barrier 61 of the tubular filter body 49 will prevent pressure within the major-through-passage 67 of the tubular filter body 49 from becoming negative. For a given pressure in the suction line 58, the amount of gas crossing the porous barrier 61 from outside the system 10 may vary depending on the amount of exhaust gas flowing into the tubular filter body 49. This should allow the exhaust system 10 to remove virtually all gas exhausted from the patient side 22.

To facilitate movement of exhaust gas from the gas exhaust orifice 46, an exhaust line 70 is shown in FIG. 1 connecting the gas exhaust orifice 46 of the housing 13 with the tubular filter inlet 52. A valve 73 may be positioned in the exhaust line 70 to regulate timing or flow of exhaust gas through the major-through-passage 67 of the tubular filter body 49. In one embodiment, the valve 73 may operate to allow exhaust gas to flow through the major-through-passage 67 of the tubular filter body 49 when the pressure on the patient side 22 of the housing 13 exceeds the pressure on the respirator side 19 of the housing 13, and/or the valve 73 may serve to prevent exhaust gas from flowing through the major-through-passage 67 of the tubular filter body 49 when the pressure on the patient side 22 of the housing 13 is less-than the pressure on the respirator side 19 of the housing 13. If the valve 73 is configured to both (a) allow exhaust gas to flow through the major-through-passage 67 when the pressure on the patient side 22 exceeds the pressure on the respirator side 19, and (b) prevent exhaust gas from flowing through the major-through-passage 67 of the tubular filter body 49 when the pressure on the patient side 22 of the housing 13 is less-than the pressure on the respirator side 19 of the housing 13, then the valve 73 may serve to keep gas in the patient side 22 until the pressure on the patient side 22 exceeds the pressure on the respirator side 19, or some other desired pressure limit.

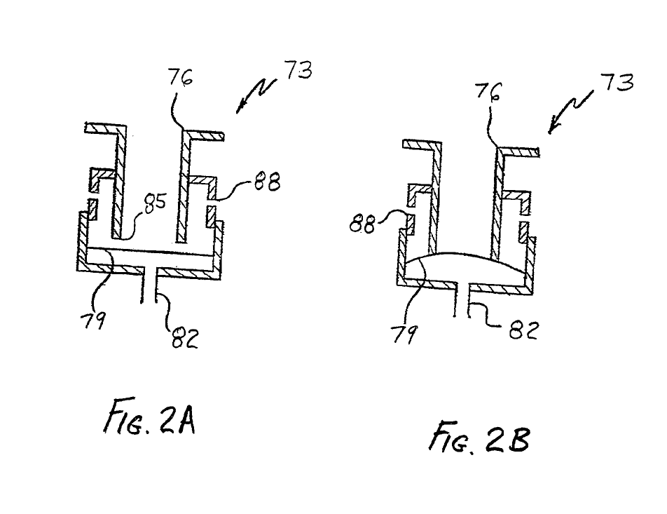

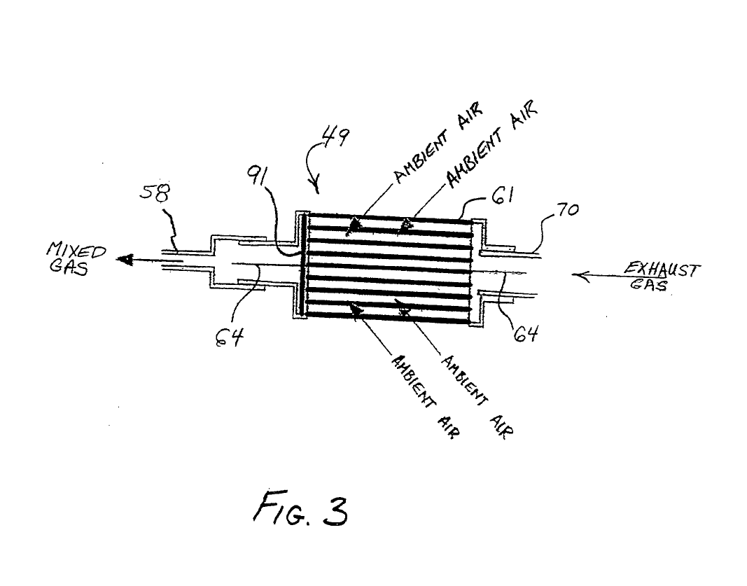

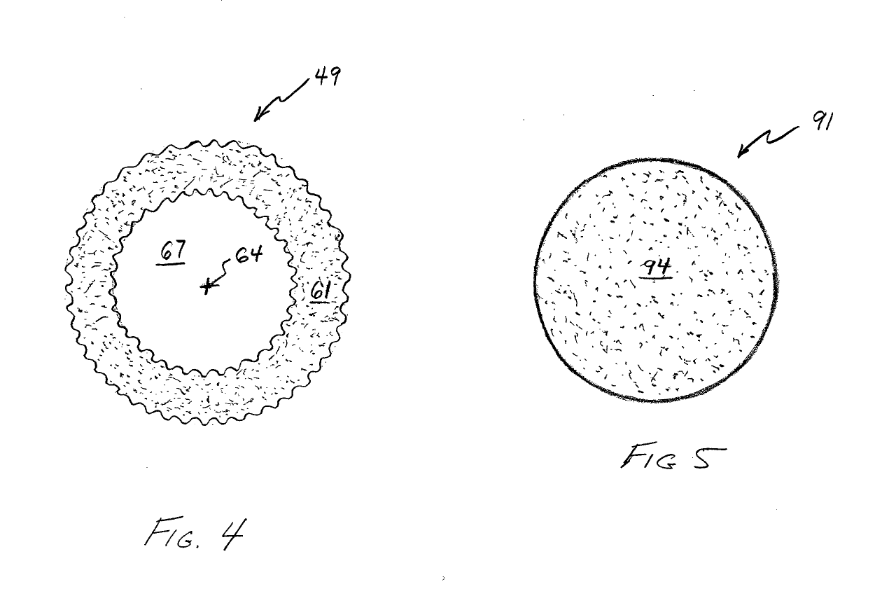

FIGS. 2A and 2B depict one type of valve 73 that may be used in the system. In FIG. 2A, the valve 73 is shown in the open position and in FIG. 2B the valve 73 is shown in the closed position. The valve 73 has an inlet tube 76, a diaphragm 79 and a pressure regulator conduit 82. The inlet tube 76 receives gas from the patient side 22. In the open position (FIG. 2A), the diaphragm 79 does not cover an end 85 of the inlet tube 76. In the open position, gas is allowed to flow from the patient side 22, through the inlet tube 76 and out the exit 88. In the closed position, (FIG. 2B), the diaphragm 79 covers the end 85 of the inlet tube 76. In the closed position, gas is prevented from flowing through the inlet tube 76 by the diaphragm 79. The diaphragm 79 may be moved between the open and closed positions by varying the pressure in the regulator conduit 82, which may be in pneumatic communication with the respirator side 19 of the housing 13, or by varying the pressure on the patient side 22, which is in communication with the inlet tube 76.

The valve 73 may be a control valve, which is capable of regulating the flow of exhaust gas through the major-through-passage 67 of the tubular filter body 49 in order to achieve a desired flow rate, or a desired pressure on the patient side 22, or both. The control valve may be operated so as to variably inhibit the flow of exhaust gas if a pressure difference between the respirator side 19 and the patient side 22 is not within a range of acceptable pressures.

A system 10 according to the invention may include another filter. FIG. 5 shows a disc-shaped exhaust gas filter 91 that is positioned to filter exhaust gas. Other shapes may be used, for example the exhaust gas filter 91 may be shaped as a parallelepiped or a cone. The exhaust gas filter 91 may be placed upstream of the tubular filter body 49. For example, the exhaust gas filter 91 may be positioned between the gas exhaust orifice 46 and the tubular filter inlet 52, and thereby filter the exhaust gas before it passes into the tubular filter body 49. In this position, the porous barrier 94 of the exhaust gas filter 91 may become laden with moisture, since the patient side 22 is expected to contain gas having a high moisture content. Once laden with moisture, the pressure drop across the exhaust gas filter 91 may be high enough to impact operation of the system 10 if the exhaust gas filter 91 is not sized properly. It is believed that the exhaust gas filter 91 may need to be sized to provide a pressure drop across the exhaust gas filter 91 that is not more than 5 centimeters of water, and preferably is less than 2 centimeters of water.

The exhaust gas filter 91 may be placed to filter exhaust gas that has passed through the major-through-passage 67 of the tubular filter body 49, for example downstream of the tubular filter body 49. If the exhaust gas filter 91 is located downstream of the tubular filter body 49, the porous barrier 94 of the exhaust gas filter 91 will filter not only exhaust gas from the patient side 22, but also gas from outside the system 10 that has crossed the porous barrier 61 of the tubular filter body 49. Therefore, when placed downstream of the tubular filter body 49, the exhaust gas filter 91 may need to be sized to handle more gas flow than when the exhaust gas filter 91 is placed upstream of the tubular filter body 49. It is believed that an average flow rate of at least five liters per minute may be needed to accommodate an average adult. Since the exhaust gas is expected to be high in moisture content relative to the gas outside the system 10, placing the exhaust gas filter 91 downstream of the tubular filter body 49 will likely mean that the gas passing through the porous barrier 94 of the exhaust gas filter 91 will be dryer than if the exhaust gas filter 91 is located upstream of the tubular filter body 49. A dryer exhaust gas filter 49 should provide a lower pressure drop for a given volume of gas crossing the porous barrier 94 of the exhaust gas filter 91.

The housing 13 may include a bias flow inlet orifice 97 on the patient side 22 of the housing 13. The bias flow inlet orifice 97 may be used to supply fresh gas or medicaments to the patient side 22 of the housing 13. For example, an inspiratory gas source 100 may provide oxygen via a controller 103 to the patient side 22. In addition, a fresh gas source may provide a therapeutic gas, or a vaporizer or a nebulizer 106 may provide a therapeutic vapor or aerosol (herein therapeutic gasses, vapors and aerosols are included in the term “medicament”) to the inspiratory gas.

Upon inhalation, the patient 28 may take the fresh gas and/or medicaments into his lungs. Exhaled gas from the patient 28 may include oxygen and/or medicaments which can be rebreathed by the patient 28 using a system according to the invention. From time to time, it will be necessary to remove some of the gas in the patient side 22 in order to allow additional fresh gas and/or a dose of medicament to enter the patient 28. However, since the patient side 22 will include exhaled gas, the patient side 22 gas may also include harmful bacteria and/or viruses. In order to prevent such contaminants from entering the suction line 58, the exhaust gas filter 91 may be a HEPA filter sized to prevent small particulates, bacteria, and/or viruses from entering the suction line 58. In some variations of the invention, the porous barrier 94 of the exhaust gas filter 91 may be selected to capture particles of medicament that have not been taken up by the patient 28.

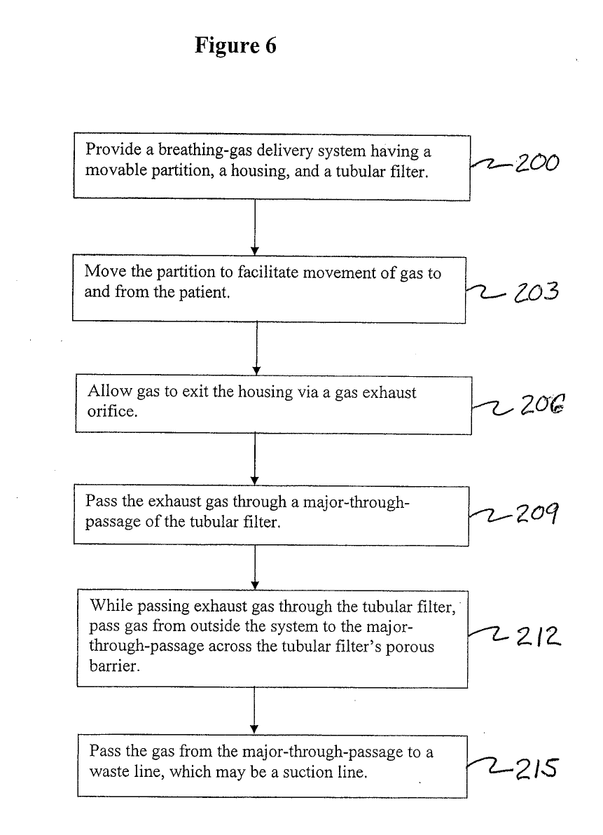

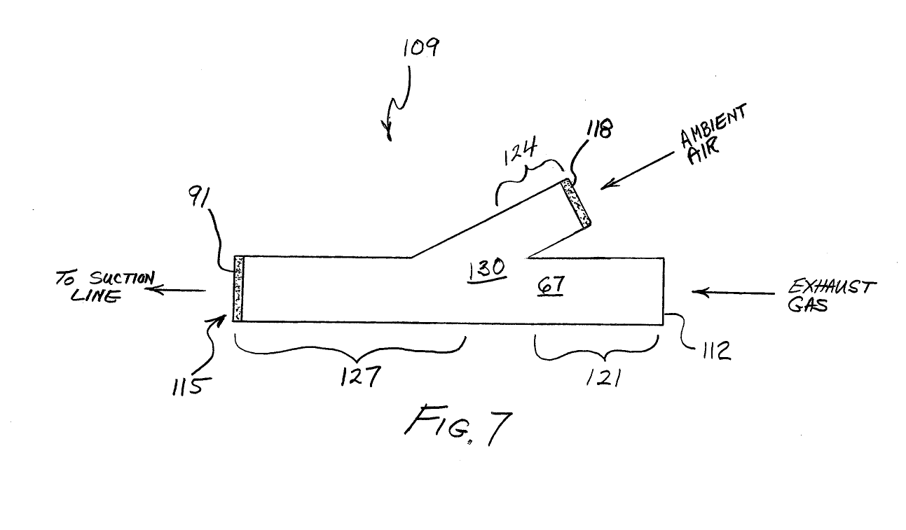

The invention may be embodied as a method of delivering breathing gas. FIG. 6 illustrates one such method. Initially, a system, like those described above, may be provided 200. The partition may be moved 203 back and forth to facilitate moving gas into and out of the patient's lungs. For example, the partition may be moved toward the patient in order to cause inspiratory gas to leave the patient side via the patient inspiration orifice to supply breathing-gas to a patient. The partition may also be moved away from the patient in order to allow the patient side of the housing to receive exhaled gas from the patient.

Movement of the partition may be effected by a respirator that is in pneumatic communication with the respirator side of the housing. Movement of the partition during inhalation may be caused by increasing the pressure on the respirator side, and movement of the partition during exhalation may be caused by decreasing the pressure on the respirator side.

If the pressure on the patient side is not at a desirable level, then gas may be allowed to leave 206 the patient side via the gas exhaust orifice, thereby providing exhaust gas to the major-through-passage. For example, if the pressure on the patient side is above the pressure on the respirator side, gas may be allowed to leave the patient side via the gas exhaust orifice. The gas leaving the patient side via the gas exhaust orifice (the “exhaust gas”) may be passed 209 through the major-through-passage.

Before, during and after passing the exhaust gas through the major-through-passage, the filter may pass 212 gas from outside the rebreather across the porous barrier to the major-through-passage. In this manner, gas from outside the system will be passing through the major-through-passage any time exhaust gas is released via the gas exhaust orifice from the patient side of the housing. The exhaust gas may also be passed through a gas exhaust filter before passing 215 into a suction line, or other disposal line that is suitable for handling and or disposing of the exhaust gas.

FIG. 7 depicts another filter body. In FIG. 7 there is shown a filter body 109 that may be used in lieu of the tubular filter body 49. The filter body 109 depicted in FIG. 7 has (a) an inlet 112 that may be connected to the gas exhaust orifice 46, (b) an outlet 115 connected to the suction line 58, and (c) a major-through-passage 67 for carrying exhaust gas from the gas exhaust orifice 46 to the suction line 58 without crossing a porous barrier 118, through which gas from outside the system 10 is allowed to move before entering the major-through-passage 67. The porous barrier 118 may be a disc-type porous barrier, similar to the exhaust gas filter 91. The filter body 109 is capable of passing gas from outside the system across the porous barrier 118 of the filter body 109 to the major-through-passage 67. The inlet 112 is shown in FIG. 7 at the beginning of a first channel 121, which may be used to convey exhaust gas. A second channel 124, also shown in FIG. 7, may be used to convey gas from outside the system. The porous barrier 118 is shown in the second channel 124.

A third channel 127 is shown extending from a merger location 130, where the first and second channels 121, 124 merge together. The third channel 127 extends from the merger location 130 toward a waste line, such as a hospital suction line 58. The third channel 127 may be used to convey gas from the first and/or second channels 121, 124 to the suction line 58.

It will now be recognized that a system 10 and method according to the invention may allow for (a) removing unwanted or excess gas from the patient side 22, which may carry a medicament and/or harmful substances, (b) preventing the pressure on the patient side 22 from becoming too high, and (c) protecting hospital personnel from infectious organisms that may be in the patient side 22.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A breathing-gas delivery system, comprising:
a movable partition;
a housing disposed about the movable partition, the housing having a respirator side on a first side of the partition, and having a patient side on a second side of the partition, and the housing having (a) a patient inspiration orifice on the patient side, adaptable to supply breathing-gas to a patient, and (b) a gas exhaust orifice on the patient side; and
a filter body having (a) an inlet connected to the gas exhaust orifice, (b) an outlet connected to a suction line, and (c) a major-through-passage for carrying exhaust gas from the gas exhaust orifice toward the suction line, and wherein the filter body is capable of passing gas from outside the rebreather across the porous barrier of the filter body to the major-through-passage.

2. The breathing-gas delivery system of claim 1, further comprising an exhaust line connecting the filter body inlet to the gas exhaust orifice of the housing.

3. The breathing-gas delivery system of claim 1, further comprising a valve positioned to regulate flow of exhaust gas through the major-through-passage of the filter body.

4. The breathing-gas delivery system of claim 3, wherein the valve allows exhaust gas to flow through the major-through-passage of the filter body when a pressure on the patient side of the housing exceeds a pressure on the respirator side of the housing.

5. The breathing-gas delivery system of claim 3, wherein the valve prevents exhaust gas from flowing through the major-through-passage of the filter body when a pressure on the patient side of the housing is less-than a pressure on the respirator side of the housing.

6. The breathing-gas delivery system of claim 3, wherein the valve is a control valve.

7. The breathing-gas delivery system of claim 6, wherein the control valve inhibits the flow of exhaust gas if a pressure difference between the respirator side and the patient side is not within a range of acceptable pressures.

8. The breathing-gas delivery system of claim 1, wherein the housing has a respirator orifice on the respirator side, the respirator orifice being in pneumatic communication with a respirator.

9. The breathing-gas delivery system of claim 1, further comprising an exhaust gas filter positioned to filter materials carried by the exhaust gas.

10. The breathing-gas delivery system of claim 9, wherein the exhaust gas filter is positioned between the gas exhaust orifice and the filter body inlet.

11. The breathing-gas delivery system of claim 9, wherein the exhaust gas filter is positioned to filter exhaust gas that has passed through the major-through passage of the filter body.

12. The breathing-gas delivery system of claim 1, further comprising a bias flow inlet orifice on the patient side of the housing.

13. The breathing-gas delivery system of claim 1, wherein the porous barrier is tubular.

14. The breathing-gas delivery system of claim 1, wherein the filter body includes a first channel, a second channel and a third channel, and wherein:
(a) the first channel is connected to receive exhaust gas from the gas exhaust orifice;
(b) the porous body is attached to the second channel;
(c) the first channel merges with the second channel at a merger location; and
(d) the third channel extends from the merger location to a waste line.

15. A method of delivering breathing-gas, comprising:
providing a breathing-gas delivery system having (a) a movable partition, (b) a housing disposed about the movable partition, the housing having a respirator side on a first side of the partition, and having a patient side on a second side of the partition, and the housing having (i) a patient inspiration orifice on the patient side, adaptable to supply breathing-gas to a patient, and (ii) a gas exhaust orifice on the patient side, and (c) a filter body having (i) an inlet connected to the gas exhaust orifice, (ii) an outlet connected to a suction line, (iii) a major-through-passage for carrying exhaust gas from the gas exhaust orifice to the suction line without crossing a porous barrier of the filter body, and (iv) capable of passing gas from outside the rebreather across the porous barrier to the major-through-passage;
moving the partition to cause inspiratory gas to leave the patient side via the patient inspiration orifice to supply breathing-gas to a patient;
allowing gas to exit the patient side via the gas exhaust orifice on the patient side to provide exhaust gas;
passing the exhaust gas through the major-through-passage; and
passing gas from outside the rebreather across the porous body to the major-through-passage;
passing the exhaust gas and the gas passed from outside the rebreather to the suction line.

16. The method of claim 15, wherein gas is allowed to exit the patient side via the gas exhaust orifice when a pressure on the patient side exceeds a pressure on the respirator side.

17. The method of claim 15, wherein moving the partition is caused by increasing a pressure in the respirator side.

18. The method of claim 15, wherein moving the partition is caused by decreasing a pressure on the respirator side.

19. The method of claim 15, further comprising providing a respirator in pneumatic communication with the respirator side, the respirator increasing a pressure in the respirator side during inspiration.

20. The method of claim 15, further comprising providing a respirator in pneumatic communication with the respirator side, the respirator decreasing a pressure in the respirator side during expiration.

21. A patient ventilator system, comprising:
a housing having a gas exhaust orifice; and
a filter body having (a) an inlet connected to the gas exhaust orifice, (b) an outlet connected to a suction line, (c) a major-through-passage for carrying exhaust gas from the gas exhaust orifice to the suction line without crossing a porous barrier of the filter body, and wherein the filter is capable of passing gas from outside the system across the porous barrier of the filter to the major-through-passage.

22. The ventilator of claim 21, wherein the porous barrier is tubular and the major through-passage is formed at least in part by the porous barrier.

23. The ventilator of claim 21, wherein the filter includes a first channel, a second channel and a third channel, and wherein:
(a) the first channel is connected to receive exhaust gas from the gas exhaust orifice;
(b) the porous body is attached to the second channel;
(c) the first channel merges with the second channel at a merger location; and
(d) the third channel extends from the merger location to a waste line.
